(19) United States
(12) Patent Application Publication
(10) Pub. No.: US 2014/0056981 A1
(43) Pub. Date: Feb. 27, 2014

(54) METHOD FOR PRODUCING DRUG-LOADED POLYMERIC NANOPARTICLES BY POLYMERIZATION IN PRESENCE OF DRUGS

(71) Applicant: CENTRO DE INVESTIGACION EN QUIMICA APLICADA, Saltillo (MX)

(72) Inventors: Raul Guillermo LOPEZ-CAMPOS, Saltillo (MX); Hened SAADE CABALLERO, Saltillo (MX)

(73) Assignee: CENTRO DE INVESTIGACION EN QUIMICA APLICADA, Saltillo (MX)

(21) Appl. No.: 13/972,722

(22) Filed: Aug. 21, 2013

Related U.S. Application Data
(60) Provisional application No. 61/691,408, filed on Aug. 21, 2012.

(51) Int. Cl. A61K 9/14; A61K 31/192

(52) U.S. Cl. CPC A61K 9/146; A61K 31/192
USPC 424/489; 514/570

(57) ABSTRACT

A method for producing drug-loaded polymeric nanoparticles, which includes the steps of: (a) preparing a first solution by dissolving a drug in a polymerizable monomer; (b) preparing a micellar solution by dissolving a surfactant and a water-soluble radical initiator in water; (c) adding said first solution to said micellar solution for polymerizing said polymerizable monomer, obtaining a dispersion of drug-loaded polymeric nanoparticles, the drug-loaded polymeric nanoparticles have a controlled size with average diameter smaller than 50 nm; and (d) evaporating residual polymerizable monomer from the dispersion of drug-loaded polymeric nanoparticles.

### TECHNICAL FIELD OF THE INVENTION

This invention relates to a method for producing nanoparticles, more particulary a method for preparing drug-loaded polymeric nanoparticles by polymerization in presence of drugs.

### BACKGROUND OF THE INVENTION

Research on drug delivery nanosystems, a non conventional via for drug administration, powerfully attracts the attention of a number of research groups around the world ((a) Thassu D, Deleers M, Pathak Y (2007) Nanoparticulate drug delivery systems: an overview. In Thassu D, Deleers M, Pathak Y (eds) Drugs and the Pharmaceutical Sciences. Nanoparticulate Drug Delivery Systems. Informa Healthcare USA, Inc, New York, pp. 1-31. (b) Jain K K (2008) Drug delivery systems-an overview. In Jain K K (ed) Methods in Molecular Biology. Drug Delivery Systems. Humana Press, Totowa, N.J., pp. 1-50. (c) Otto D P, de Villiers M M (2009) Physicochemical principles of nanosized drug delivery system. In de Villiers M M, Aramwit P, Kwon G S (eds) Biotechnology: Pharmaceutical Aspects. Nanotechnology in Drug Delivery. Springer, New York, pp. 3-33. (d) D'Mello S R, Das S K, Das N G (2009) Polymeric nanoparticles for small-molecule drug: biodegradation of polymers and fabrication of nanoparticles. In Pathak Y, Thassu D (eds) Drug Delivery Nanoparticles Formulation and Characterization. Informa Healthcare USA, Inc, New York, pp. 16-34. (e) Surendiran A, Sandhiya S, Pradhan S C, Adithan C (2009) Novel Applications of Nanotechnology. Medicine Indian J Med Res 130: 689-701)). Attractiveness of these systems comes from the possibility they offer for drugs efficacy enhancement and reduction of the undesirable side effects in the treatment of cancer, diabetes, infections and others. The firsts reports on drug delivery micro and nano systems appeared some few decades ago (Khanna S C, Speiser P (1969) Epoxy resin beads as pharmaceutical dosage form I: methods of preparation. J Pharm Sci 58: 1114-1117; and Brasseur F, Couvreur P, Kante B, Deckers-Passau L, Roland M, Deckers C, Speisers P (1980) Actinomycin D adsorbed on polymethylcyanoacrylate nanoparticles: increased efficiency against an experimental tumor. Eu. J Cancer 16: 1441-1445). Since, a number of reports on this subject have been published in the specialized literature (Otto D P, de Villiers M M (2009) Physicochemical principles of nanosized drug delivery system. In de Villiers M M, Aramwit P, Kwon G S (eds) Biotechnology: Pharmaceutical Aspects. Nanotechnology in Drug Delivery. Springer, New York, pp. 3-33).

Among the distinctive features of drug delivery nanosystems that makes them so interesting are their increasing capacity for hydrophobic drugs dissolution; drug protection once inside the organism; enhanced specific targeting; better drug delivery control; and, generation of alternative routes for destroying malign cells and microorganisms that have developed drug resistance.

In addition to drug-loaded polymeric nanoparticles there is a variety of drug delivery nanosystems, such as liposomes, fullerenes, nanotubes, quantum dots, polymer-coated magnetic nanoparticles, and dendrimers (Surendiran A, Sandhiya S, Pradhan S C, Adithan C (2009) Novel Applications of Nanotechnology. Medicine Indian J Med Res 130: 689-701). While all of these systems allow flexibility in choosing the type of drug to be entrapped, polymeric nanoparticles have several advantages that make them one of the most promising options for the developing of drug delivery nanosystems. Some of these advantages are an enhanced stability of the loaded nanostructures; higher capacity for drug loading; increased drug protection; feasibility of modification or functionalization of their surface; and, the possibility of choosing the type and characteristics of the polymers to be used for a better drug release control (D'Mello S R, Das S K, Das N G (2009) Polymeric nanoparticles for small-molecule drug: biodegradation of polymers and fabrication of nanoparticles. In Pathak Y, Thassu D (eds) Drug Delivery Nanoparticles Formulation and Characterization. Informa Healthcare USA, Inc, New York, pp. 16-34).

Polymeric nanoparticles with dissolved, entrapped, encapsulated or attached drugs are prepared by a number of methods, which usually make use of preformed polymers or polymers synthesized during the manufacturing process. The reported methods for preparing drugs containing polymeric nanoparticles based on the use of preformed polymers usually include the preparation of a drug solution, which is dispersed as nanodroplets in a continuous phase using surfactants and a combination of high-speed homogenization and sonication; the polymer can be dissolved either in the dispersed or the continuous phase. Some of the methods used for preparing drug-loaded polymeric nanoparticles are nanonization (Kraehenbuhl J P, Neutra M R (2000) Epithelial M cells: Differentiation and Function. Annual Review of Cell Dev Biol 16: 301-332); nanoprecipitation (Stella B, Arpicco S, Rocco F, Marsaud V, Renoir J M, Cattel L, Couvreur P (2007) Encapsulation of gemcitabine lipophilic derivatives into polycyanoacrylate nanospheres and nanocapsules. I J Pharmaceut 344: 71-77; and Alves Pereira M, Furtado Mosqueira V C, CarneiroVilela J M, Spangler Andrade M, Andrade Ramaldes G, Nascimento Cardoso V (2008) PLA-PEG nanocapsules radiolabeled with ^{99m}Technetium-HMPAO: Release properties and physicochemical characterization by atomic force microscopy and photon correlation spectroscopy. Eur J Pharm Sci 33: 42-51); emulsion-diffusion (Hyang H J, Hyeon Y L, You S G, Jin-Chul K (2008) Colloidal stability and in vitro permeation study of poly(□-caprolactone) nanocapsules containing hinokitiol. J Ind Eng Chemistry 14: 608-613; and Mi-Jung Ch, Apinan S, Onanong N, Sang-Gi M, Uracha R (2009) Physical and light oxidative properties of eugenol encapsulated by molecular inclusion and emulsion-diffusion method. Food Res. Int 42: 148-156); double emulsification (Young-Il J, Hee-Sam N, Dong-Hyuk S, Dong-Gon K, Hyun-Chul L, Mi-Kyeong J, Sang-Kwon N, Sung-Hee R, Sun-Il K, Jae-Woon N (2008) Ciprofloxacin-encapsulated poly(dl-lactide-co-glycolide) nanoparticles and its antibacterial activity. Int J Pharmaceut 352: 317-323); emulsion-coacervation (Lertsutthiwong P, Rojsitthisak P, Nimmannit U (2009) Preparation of turmeric oil-loaded chitosan-alginate biopolymeric nanocapsules. Mat Sci Eng C 29: 856-860); polymer-coating (Chen Y, Lin X, Park H, Greever R (2009) Study of artemisinin nanocapsules as anticancer drug delivery systems. Nanomedicine: NBM 5: 316-322); layer-by-layer (Tartaj P, Morales M P, Veintemillas-Verdaguer S, González-Carreño T, Serna, C J (2003) The preparation of magnetic nanoparticles for applications in biomedicine. J Phys D Appl Phys 36R182); and solvent evaporation (desRieux A, Fievez V, Garinot M, Schneider Y J, Préat V (2006) Nanoparticles as potential oral delivery systems of proteins and vaccines: A mechanistic approach. J Cont Rel 116: 1-27). Through these methods, nanoparticles with mean diameters from 50-100 nm are usually prepared, however, they are not suitable for the preparation of smaller-size particles. In fact, to our best knowledge, there are no reports describing the manufacture of drug-loaded polymeric nanoparticles with sizes smaller than 50 nm. Although there does not exist a clear definition of ultrafine nanoparticles, in the case of drug delivery systems using carrying nanoparticles, this term could be used to name particles in the range of less than 10 to 50 nanometers. Such particles possess a huge area to volume ratio, which makes them very attractive for preparing drug-loaded polymeric nanoparticles, where the drugs are attached on the particle surface and for surface functionalizing in active targeting. Moreover, this feature of nanoparticles along with their lightness in weight leads to an enhanced stability of the ultrafine nanoparticles during storage. The lack of published results on the topic is noteworthy, as one would expect the advantages of the known larger nanoparticles to be enhanced by decreasing their size. For instance, their smallness allows to reduce their clearance by the reticuloendothelial system (Thassu D, Deleers M, Pathak Y (2007) Nanoparticulate drug delivery systems: an overview. In Thassu D, Deleers M, Pathak Y (eds) Drugs and the Pharmaceutical Sciences. Nanoparticulate Drug Delivery Systems. Informa Healthcare USA, Inc, New York, pp. 1-31) and even to pass through cell membrane beneath a critical size (50 nm) (Logothetidis S (2006) Nanotechnology in Medicine: The medicine of tomorrow and nanomedicine. Hippokratia 10: 7-21). Another interesting advantage of drug-loaded nanoparticles with diameters smaller than 50 nm is that they would be able to cross through intestinal walls to enter the blood stream (desRieux A, Fievez V, Garinot M, Schneider Y J, Préat V (2006) Nanoparticles as potential oral delivery systems of proteins and vaccines: A mechanistic approach. J Cont Rel 116: 1-27; and Jani P, Halbert G W, Langridge J, Florence A T (1990) Nanoparticle uptake by the rat gastrointestinal mucosa: quantitation and particle size dependency. J Pharm Pharmacol 42: 821-826). Furthermore, nanoparticles smaller than 20 nm can pass through the pores of blood vessels as they circulate throughout the body (Logothetidis S (2006) Nanotechnology in Medicine: The medicine of tomorrow and nanomedicine. Hippokratia 10: 7-21).

### SUMMARY OF THE INVENTION

The object of the invention is to provide a method for producing drug-loaded polymeric nanoparticles, the method includes the steps of: (a) preparing a first solution by dissolving a drug in a polymerizable monomer; (b) preparing a micellar solution by dissolving a surfactant and a water-soluble radical initiator in water; (c) adding the first solution to the micellar solution for polymerizing the polymerizable monomer, obtaining a dispersion of drug-loaded polymeric nanoparticles, the drug-loaded polymeric nanoparticles have a controlled size with average diameter smaller than 50 nm; and (d) evaporating residual polymerizable monomer from the dispersion of drug-loaded polymeric nanoparticles.

Another object of the invention is to provide a drug-loaded polymeric nanoparticle produced by the above method.

### BRIEF DESCRIPTION OF THE FIGURES

The characteristic details of the invention are described in the following paragraphs together with the attached drawings, with the purpose of defining the invention, but without limiting its range.

FIG. 1 shows QLS-particle size distributions for Ibuprofen-loaded nanoparticles from two embodiments of polymerization: Example 1 (a) and Example 2 (b) according to the invention.

FIG. 2 shows STEM micrographs and the corresponding particle size histograms of samples of Ibuprofen-loaded nanoparticles from two embodiments of polymerization: Example 1 (a) and Example 2 (b) according to the invention.

FIG. 3 shows thermograms obtained by DSC of samples of the Ibuprofen-loaded nanoparticles from two embodiments of polymerization: Example 1 (a) and Example 2 (b) according to the invention.

### DETAILED DESCRIPTION OF THE INVENTION

The following description is only intended to represent the way of how the principles of the invention can be implemented in various embodiments. The embodiments described herein do not intend to be a comprehensive representation of the invention. The following embodiments are not to limit the invention to the precise form published in the following detailed description.

The terms “drug” as used herein refer to a medicinal material, a compound or a mixture thereof, suitable and medically indicated for treatment of a malcondition in a patient. The drug can be in a solid physical form or a liquid physical form at about room temperature or at about body temperature, depending on the melting point of the material.

### Preparation of Drug-Loaded Polymeric Nanoparticles

We present here a new approach for preparing drug-loaded polymeric nanoparticles by polymer formation in the presence of drugs. The method includes the following steps: (a) preparing a first solution by dissolving a drug in a polymerizable monomer; (b) preparing a micellar solution by dissolving a surfactant and a water-soluble radical initiator in water; (c) adding the first solution to the micellar solution for polymerizing the polymerizable monomer, obtaining a dispersion of drug-loaded polymeric nanoparticles, where the drug-loaded polymeric nanoparticles have a controlled size with average diameter smaller than 50 nm; and (d) evaporating residual polymerizable monomer from the dispersion of drug-loaded polymeric nanoparticles. The drugs, either liquid or solid, must be soluble in the polymerizable monomer and inert to the polymerizable monomer and initiator. Polymerization temperature must be lower than the drug decomposition temperature. At the end of the polymerization, nanoparticles with average diameters smaller than 50 nm, usually between 5 and 40 nm, dispersed in a continuous phase are obtained. These nanoparticles are composed of polymer, drugs and residual monomer. Then, the obtained dispersion is subjected to heating and or vacuum for evaporating the residual monomer, avoiding the use of high temperatures in order to protect the drugs from thermal decomposition. During this step, it could be necessary to add an extra amount of water in order to replace that may have evaporated with the solvent. Once all the residual monomer is removed, a dispersion of drug-loaded polymeric nanoparticles with average diameters smaller than 50 nm is obtained. Subsequently, these nanoparticles can be suitably functionalized to increase their biocompatibility.

### Step (a): Preparation of the First Solution by Dissolving a Drug in a Polymerizable Monomer

Drugs to be loaded in the polymeric nanoparticles, include but are not limited to, drug molecules, peptides/proteins, imaging agents, genetic material or a combination of these compounds. The drugs can be organic compounds that are poorly soluble or insoluble in water but readily soluble in organic solvents. The drugs agent is added to the polymeric dispersion either in the form of dry powder or as a solution in dichloromethane, chloroform, ethanol or ether depending on the solubility of the drug in that solvent to form an optically clear solution. Examples of such drugs include, but are not limited to, antineoplastic agents such as Paclitaxel, Docetaxel, Rapamycin, Doxorubicin, Daunorubicin, Idarubicin, Epirubicin, Capecitabine, Mitomycin C, Amsacrine, Busulfan, Tretinoin, Etoposide, Chlorambucil, Chlormethine, Melphalan, and Benzylphenylurea (BPU) compounds; phytochemicals and other natural compounds such as curcumin, curcuminoids, and other flavinoids; steroidal compounds such as natural and synthetic steroids, and steroid derivatives like cyclopamine; antiviral agents such as Aciclovir, Indinavir, Lamivudine, Stavudine, Nevirapine, Ritonavir, Ganciclovir, Saquinavir, Lopinavir, Nelfinavir; antifungal agents such as Itraconazole, Ketoconazole, Miconazole, Oxiconazole, Sertaconazole, Amphotericin B, and Griseofulvin; antibacterial agents such as quinolones including Ciprofloxacin, Ofloxacin, Moxifloxacin, Methoxyfloxacin, Pefloxacin, Norfloxacin, Sparfloxacin, Temafloxacin, Levofloxacin, Lomefloxacin, Cinoxacin; antibacterial agents such as penicillins including Cloxacillin, Benzylpenicillin, Phenylmethoxypenicillin; antibacterial agents such as aminoglycosides including Erythromycin and other macrolides; antitubercular agents such as rifampicin and rifapentin; and anti-inflammatory agents such as Ibuprofen, Indomethacin, Ketoprofen, Naproxen, Oxaprozin, Piroxicam, Sulindac. Preferably, the drug loaded in the nanoparticles is in a range from 1% to 50% by weight of the polymerizable monomer-drug mixture.

Specific examples of the polymerizable monomers for use in preparing the nanoparticles of the invention include mono- or poly-functional vinyl monomers, which can be radically polymerized.

Specific examples of the monofunctional polymerizable monomers include styrene derivatives such as styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, and p-phenylstyrene; acrylic monomers such as methyl acrylate, ethyl acrylate, n-propyl acrylate, iso-propyl acrylate, n-butyl acrylate, iso-butyl acrylate, tert-butyl acrylate, n-amyl acrylate, n-hexyl acrylate, 2-ethylhexyl acrylate, n-octyl acrylate, n-nonyl acrylate, cyclohexyl acrylate, benzyl acrylate, dimethylphosphate ethyl acylate, diethylphosphate ethyl acylate, dibutylphosphate ethyl acylate, and 2-benzoyloxyethyl acrylate; methacrylic monomers such as methyl methacrylate, ethyl methacrylate, n-propyl methacrylate, iso-propyl methacrylate, n-butyl methacrylate, iso-butyl methacrylate, tert-butyl methacrylate, n-amyl methacrylate, n-hexyl methacrylate, 2-ethylhexyl methacrylate, n-octyl methacrylate, n-nonyl methacrylate, diethylphosphate ethyl methacylate, dibutylphosphate ethyl methacylate; vinyl esters such as methylenealiphaticmonocarboxylic acid esters, vinyl acetate, vinyl propionate, vinyl butyrate, vinyl benzoate, vinyl formate; vinyl ethers such as vinyl methyl ether, vinyl ethyl ether, and vinyl isobutyl ether; vinyl ketones such as vinyl methyl ketone, vinyl hexyl ketone, vinyl isopropyl ketone, and combinations thereof.

Specific examples of the polyfunctional polymerizable monomers include diethylene glycol diacrylate, triethylene glycol diacrylate, tetraethylene glycol diacrylate, polyethylene glycol diacrylate, 1,6-hexanediol diacrylate, neopentyl glycol diacrylate, tripropylene glycol diacrylate, polypropylene glycol diacrylate, 2,2′-bis{4-(acryloxydiethoxy)phenyl}propane, trimethylolpropane triacrylate, tetramethylolmethane tetraacrylate, ethylene glycol dimethacrylate, diethylene glycol dimethacrylate, triethylene glycol dimethacrylate, tetraethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2,2′-bis{4-(methacryloxydiethoxy)phenyl}propane, 2,2′-bis{4-(methacryloxypolyethoxy)phenyl}propane, trimethylolpropane trimethacrylate, tetramethylolmethane tetramethacrylate, divinyl ether, and combinations thereof.

The monofunctional polymerizable monomers mentioned above can be used alone or in combination. In addition, polyfunctional polymerizable monomers can be used together with one or more of monofunctional monomers. Among the monomers mentioned above, styrene, (meth)acrylic acid and/or their derivatives are preferably used alone or in combination with other monomers.

The preferred polymerizable monomer is methyl methacrylate. The polymerizable monomer may be incorporated in the first solution in an amount of 50% to 99% by weight based on the total weight of the first solution.

### Step (b): Preparation of a Micellar Solution by Dissolving a Surfactant and a Water-Soluble Radical Initiator in Water

The size and stability of the nanoparticles are dependent upon the amount of water loading and the molar ratio of water to surfactant. The surfactant used to disperse the polymerizable monomer may be nonionic, anionic or cationic in kind. The surfactants may be anionic, but are not limited to: for example, sodium dodecylsulfate, salts of fatty acids, such as salts of dialkylsulfosuccinic acid, especially sodium bis(2-ethylhexyl)sulfosuccinate, salts of alkyl and aryl sulfonates and salts of tri-chain amphiphilic compounds, such as sodium trialkyl sulfo-tricarballylates. The anionic surfactants may also comprise hydrophilic non-ionic functionalities, such as ethylene oxide or hydroxyl groups. They may be nonionic: for example, polyoxyethylene alkyl ethers, acetylene diols and their derivatives, alkylthiopolyacrylamides, copolymers of polyoxyethylene and polyoxypropylene, alcohol alkoxylates, sugar-based derivatives; they may be cationic, such as alkyl amines, quaternary ammonium salts; or they may be amphoteric: for example, betaines. However the surfactant should normally be selected such that it is either uncharged (non-ionic), has no overall charge (amphoteric or zwitterionic surfactant) or matches the charge of the stimulus-responsive polymer used. The preferred are sodium dodecylsulfate and sodium bis(2-ethylhexyl)sulfosuccinate. The surfactants may be incorporated in the micellar solution in an amount of 0.1% to 15%, preferably 0.5% to 12.5%, in particular 1% to 10%, by weight based on the total weight of the micellar solution.

The polymerization of the step (c) may be initiated using a charged or chargeable water-soluble radical initiator species, such as, for example, a salt of the persulfate anion, especially potassium persulfate, or with a neutral initiator species if a charged or chargeable co-monomer species is incorporated in the preparation. The initiation of the radical polymerization may then triggered by the decomposition of the initiator resulting from exposure to heat or to light. In the case of initiation using heat, a reduced temperature can be used by combining the initiator compound, such as potassium persulfate, with an accelerator compound, such as sodium metabisulfite. The water-soluble radical initiator is used preferably in an amount of 0.01 to 10%, preferably 0.05% to 7.5%, in particular 0.1% to 5%, by weight based on the total weight of the micellar solution.

In an alternative embodiment of the invention, a chain transfer agent may be added to the micellar solution. The chain transfer agent is a molecule, which is known to reduce molecular weight during a free-radical polymerization via a chain transfer mechanism. These agents may be any thiol-containing molecule and can be either monofunctional or multifunctional. The chain transfer agent may be hydrophilic, hydrophobic, amphiphilic, anionic, cationic, neutral, zwitterionic or responsive. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis(borondifluorodimethyl-glyoxirnate) (CoBF) may also be used. Suitable thiols include but are not limited to C2-C18 alkyl thiols such as dodecane thiol, thioglycolic acid, thioglycerol, cysteine and cysteamine. Thiol-containing oligomers or polymers may also he used such as poly(cysteine) or an oligomer or polymer which has been post-functionalised to give a thiol group(s), such as polyethyleneglycol) (di)thio glycollate, or a pre-formed polymer functionalised with a thiol group, for example, reaction of an end or side-functionalised alcohol such as poly(propylene glycol) with thiobutyrolactone, to give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerization including alkyl halides and transition metal salts or complexes. More than one chain transfer agent may be used in combination. When the chain transfer agent is providing the necessary hydrophilicity in the copolymer, it is preferred that the chain transfer agent is hydrophilic and has a molecular weight of at least 1000 Daltons.

Hydrophilic chain transfer agents typically contain hydrogen bonding and/or permanent or transient charges. Hydrophilic chain transfer agents include but are not limited to thio-acids such as thioglycolic acid and cysteine, thioamines such as cysteamine and thio-alcohols such as 2-mercaptoethanol, thioglycerol and ethylene glycol mono- (and di-)thio glycollate. Hydrophilic macro-CTAs (where the molecular weight of the CTA is at least 1000 Daltons) can be prepared from hydrophilic polymers synthesised by RAFT (or MADIX) followed by reduction of the chain end, or alternatively the terminal hydroxyl group of a preformed hydrophilic polymer can be post functionalised with a compound such as thiobutyrolactone.

Hydrophobic chain transfer agents include but are not limited to linear and branched alkyl and aryl(di)thiols such as dodecanethiol, octadecyl mercaptan, 2-methyl-1-butanethiol and 1,9-nonanedithiol.

The chain transfer agent is used preferably in an amount of 0.01 to 10%, preferably 0.05% to 7.5%, in particular 0.1% to 5%, by weight based on the total weight of the micellar solution.

### Step (c): Polymerization in the Presence of Drugs by Adding the First Solution to the Micellar Solution

The polymerization of the polymerizable monomers in presence of drugs is carried out by adding (in a dosified manner) the first solution prepared in the step (a) to the micellar solution prepared in the step (b). At slow enough dosing rate of monomer and sufficient surfactant to stabilize the nanoparticles, this technique allows obtaining drug-loaded polymeric nanoparticles with average diameters smaller than 50 nm, usually between 5 and 40 nm, dispersed in a continuous phase. In accordance with the emulsion polymerization theory when the polymerization is carried out in semicontinuous fashion, a slow enough rate of monomer dosing allows the formation of new particles instead of the growth of those already existent. Eventually, this leads to dispersions composed of a great number of very small polymeric nanoparticles. When a solution monomer-drug instead of neat monomer is dosed, the drug is entrapped in the polymeric platform due to strong hydrophobicity of the former. The difference with the method in which the polymeric nanoparticles are first prepared and then loaded with an organic solution containing the drug is the greater simplicity of the method that is now described.

Step (d): Evaporation of Residual Polymerizable Monomer from the Dispersion of Drug-Loaded Polymeric Nanoparticles

The obtained dispersion is subjected to heating and or vacuum for evaporating the residual monomer, avoiding the use of high temperatures in order to protect the drugs from thermal decomposition. During this step, it could be necessary to add an extra amount of water in order to replace that may have evaporated along the monomer. Once all the residual monomer is removed, a dispersion of drug-loaded polymeric nanoparticles with average diameters smaller than 50 nm is obtained.

The nanoparticles containing at least one active ingredient or drug or a combination of active ingredients or drugs prepared by the above described method (e.g., nanoparticles with entrapped active ingredients or drugs) may be used for the treatment of pathological conditions arising out of various diseases including but not limited to cancer, inflammation, infection and neurodegeneration.

A benefit of the present invention is the ability to generate polymeric nanoparticles of controlled size and composition, where the size of particles in a population can be substantially homogeneous. The polymeric nanoparticles of the present invention comprise an entrapped substantially pure active ingredient or drug. It will be understood by one of skill in the art that two or more active ingredients can be co-formulated, in which case the purity shad refer to the combined active agents.

### Examples of the Invention

The invention will now be described with respect to the following examples, which are solely for the purpose of representing the way of carrying out the implementation of the principles of the invention. The following examples are not intended to be a comprehensive representation of the invention, or try to limit the scope thereof.

The polymerizations in presence of Ibuprofen were carried out in a 150-mL jacketed glass reactor equipped with a reflux condenser and mechanical stirring (650 rpm). The procedure was as follows. The required quantities of sodium dodecyl sulfate, sodium bis(2-ethylhexyl)sulfosuccinate and potassium persulfate were dissolved in the total amount of water (see recipe in Table 1) to prepare a micellar solution and charged into a reactor. This solution was heated to 70° C. and bubbled with argon for 1 h. Then, a solution of Ibuprofen-methyl methacrylate, at two drug concentrations 20% by weight (Example 1) and 25% by weight (Example 2) were fed at a rate of 0.07 g/min using a KD Scientific syringe pump. Once the solution addition was completed (8 h), a sample of the final latex was taken to determine conversion by gravimetry. Then, a part of the dispersion was filtered through a 0.2 μm filter and samples of the filtered were taken for characterization. The remaining dispersion was heated at 40° C. under reduced pressure to evaporate the unreacted monomer.

TABLE 1

| Component | Example 1 | Example 2 |
|---|---|---|
| Water (g) | 93.2175 | 93.1286 |
| Sodium dodecyl sulfate (g) | 3.6743 | 3.6735 |
| sodium bis (2-ethylhexyl) | 1.2248 | 1.2245 |
| sulfosuccinate (g) | | |
| Potassium persulfate (g) | 0.0495 | 0.0408 |
| Methyl methacrylate (g) | 26.8599 | 25.8222 |
| Ibuprofen (g) | 6.7149 | 8.6074 |

For the quantification of Ibuprofen content in polymeric nanoparticles, the dried solid resulting from solid content determination was analyzed in a Shimadzu multiespec-1501UV-vis spectrophotometer. For this, a calibration curve was elaborated ranging from 50 to 500 ppm of Ibuprofen by dissolving the required quantities of the drug in chloroform HPLC grade and reading the absorbance at 272 nm. The analysis of the samples required the dissolution of 0.5 g of the product in 10 mL of chloroform grade HPLC. Z-average particle size (D_{z}) was measured at 25° C. by quasielastic light scattering (QLS) in a Malvern Zetasizer Nano-ZS90 apparatus. To eliminate multiple scattering and particle interactions, latexes were diluted 50 times with water. The average size and distribution of the particle size was determined from measurements in a JEOL JSM-7401F scanning-transmission electron microscope (STEM). The samples were prepared by mixing one latex drop with 10 g of water. Then, one drop of this dispersion was deposited on a copper grid and allowed to dry. The diameter of around 700 particles were measured from the micrographs to obtain D_{w}, Dₙ and PDI (D_{w}/Dₙ), being D_{w} and Dₙ the weight- and number-average diameters and PDI the polydispersity index, which were calculated using the following equations:

$$\begin{matrix}{D}_{n} = \frac{{\sum }_{i} {n}_{i} {D}_{i}}{{\sum }_{i} {n}_{i}} = \frac{\sum {n}_{i} {D}_{i}}{n} & ( 1 ) \\ {D}_{w} = \frac{{\sum }_{i} {n}_{i} {D}_{i}^{4}}{{\sum }_{i} {n}_{i} {D}_{i}^{3}} & ( 2 )\end{matrix}$$

where nᵢ is the number of particles of size Dᵢ and n is the total number of measured particles.

Glass transition temperature (Tg) determination was carried out in a modulated differential scanning calorimeter (DSC) TA Instruments Q200. Measurement was performed at a heating rate of 10° C./min in the range 0 to 170° C., under a nitrogen flow of 50 mL/min. The glass transition temperature was evaluated by analyzing the reversible heat flow signal using the criteria of half-height.

The final dispersions obtained at the end of the polymerizations carried out using Example 1 (20% by weight of Ibuprofen) and Example 2 (25% by weight of Ibuprofen) show a translucent appearance, similar to that obtained in the semicontinuous heterophase polymerizations of methyl methacrylate. The observation suggests that the latexes are composed of very small poly(methyl methacrylate) nanoparticles, containing probably all the Ibuprofen used in the formulation.

The values of the solids content in the latexes before and after filtration using 0.2 μm filters, determine the fate of the Ibuprofen dissolved in the polymerizable monomer and added during the polymerizations. Because the water solubility of Ibuprofen is very low (0.37 mg/mL at 25° C.), the amount of drug dissolved in the aqueous phase at the end of polymerizations would be negligible, so Ibuprofen would be located either within the polymer particles and/or dispersed in the aqueous phase. Its presence within micelles can be discarded because the absence of free surfactant in the latex at the end of a semicontinuous heterophase polymerizations. The way in which the filtration operation allows to determine where Ibuprofen is allocated in the final latexes is explained below. First, the 0.2 gm filter would retain all Ibuprofen aggregates with sizes greater than 200 nm dispersed in the aqueous phase, outside the nanoparticles, causing the total solids content in the filtered latex to be lower than that of the latex before filtration. On the contrary, if no difference is determined between the total solids content in the latex before and after filtration, the presence of Ibuprofen aggregates with sizes greater than 200 nm in the aqueous phase can be discarded. However, in this latter case, the coexistence of Ibuprofen-loaded poly(methyl methacrylate) nanoparticles and Ibuprofen aggregates dispersed in the aqueous phase, both with sizes ≦200 nm, cannot be over ruled. A further measurement of the filtered latex by QLS will determine if only Ibuprofen-loaded poly(methyl methacrylate) nanoparticles exist or there are also Ibuprofen aggregates. An intensity-average diameter (D_{z}) of particle smaller than 50 nm (that expected for poly(methyl methacrylate) nanoparticles) and only one population in the particle size distribution given by the apparatus would indicate that only Ibuprofen-loaded poly(methyl methacrylate) nanoparticles exist, discarding the presence of Ibuprofen aggregates with sizes ≦200 nm outside the nanoparticles. In this case, as the existence of Ibuprofen aggregates >200 nm has previously been ruled out, the conclusion would be that all Ibuprofen added along methyl methacrylate was incorporated into the poly(methyl methacrylate) nanoparticles. However, if another population in addition to that corresponding to Ibuprofen-loaded poly(methyl methacrylate) nanoparticles appears in the particle size distribution given by the apparatus, it would mean that Ibuprofen aggregates with sizes 200 nm coexist with the loaded polymeric nanoparticles. Consequently, the conclusion would be that at least part of the added Ibuprofen was not incorporated into the poly(methyl methacrylate) nanoparticles. Returning to the case where the QLS measurement of the filtered latex gives only one particle size distribution, which is assigned to the Ibuprofen-loaded poly(methyl methacrylate) nanoparticles population, emerges the question of why cannot also exist a population of Ibuprofen aggregates with a similar size distribution to that of Ibuprofen-loaded poly(methyl methacrylate) nanoparticles. Here, it would be impossible to distinguish between both populations. However, the occurrence probability of this situation is practically insignificant because the difference between the mechanism originating the particles and that forming the hypothetical aggregates.

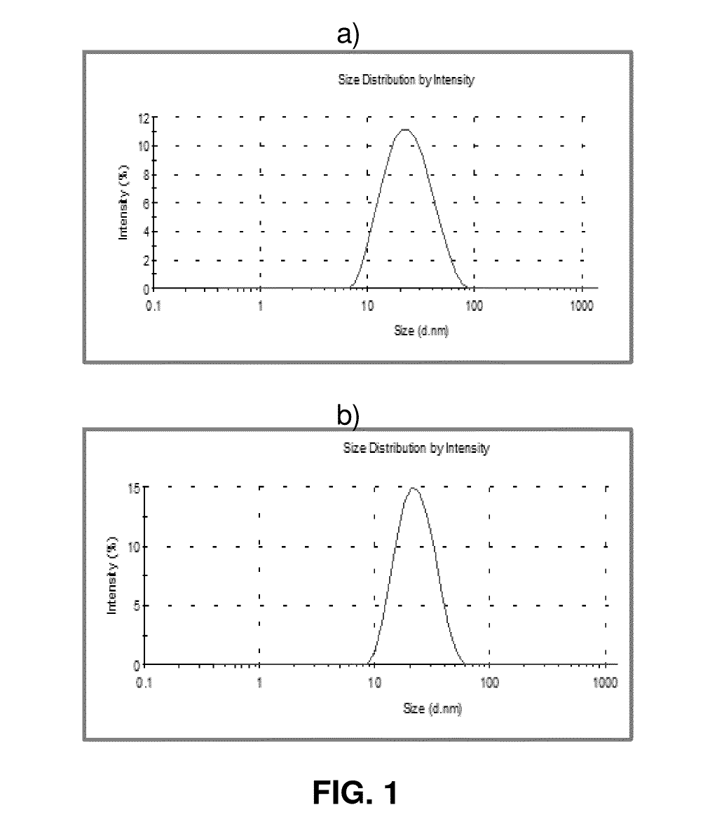

Table 2 shows the solids content in the latex before and after the filtration for both polymerizations. Data in this table indicate that the solids content values in the latexes before and after filtration are practically the same for both polymerizations. In accordance with the previous paragraph, these results indicate that there were no Ibuprofen aggregates larger than 200 nm in either dimension outside the nanoparticles. In consequence, it can be said that all the added Ibuprofen along methyl methacrylate was inside the nanoparticles and/or forming aggregates equal or smaller than 200 nm in size. The particle size distributions obtained by QLS for the filtered latexes from both polymerizations are shown in FIG. 1. Both curves in this FIG. 1 display only one particle population, with D_{z} values of 27.0 and 33.7 nm for Example 1 and Example 2 latexes, respectively, which would correspond to Ibuprofen-loaded poly(methyl methacrylate) nanoparticles. This discards the presence of Ibuprofen aggregates with size equal or smaller than 200 nm in either dimension, demonstrating that all the Ibuprofen in the latexes was inside the nanoparticles.

TABLE 2

| Run | Example 1 | Example 2 |
|---|---|---|
| Conversion (%) | 91.30 | 86.68 |
| Solids content before | 27.47 | 27.11 |
| filtration (%) | | |
| Solids content after | 27.54 | 27.11 |
| filtration (%) | | |
| Dₙ (nm) | 19.21 | 15.90 |
| PDI | 1.15 | 1.14 |
| Theoretical Ibuprofen | 18.6 | 23.9 |
| content (%) | | |
| Ibuprofen content by UV-Vis | 15.7 | 23.1 |
| spectroscopy (%) | | |

The solids content in the final latexes determined in the inquiry on the fate of Ibuprofen, allow to highlight that our method is able to produce dispersions with relatively high drug-loaded nanoparticles contents. The values in this study (≈27%) are unusually high compared with those obtained in the most of the works on drug-loaded polymeric nanoparticles, where the high solids contents typically range from 10 to 20%.

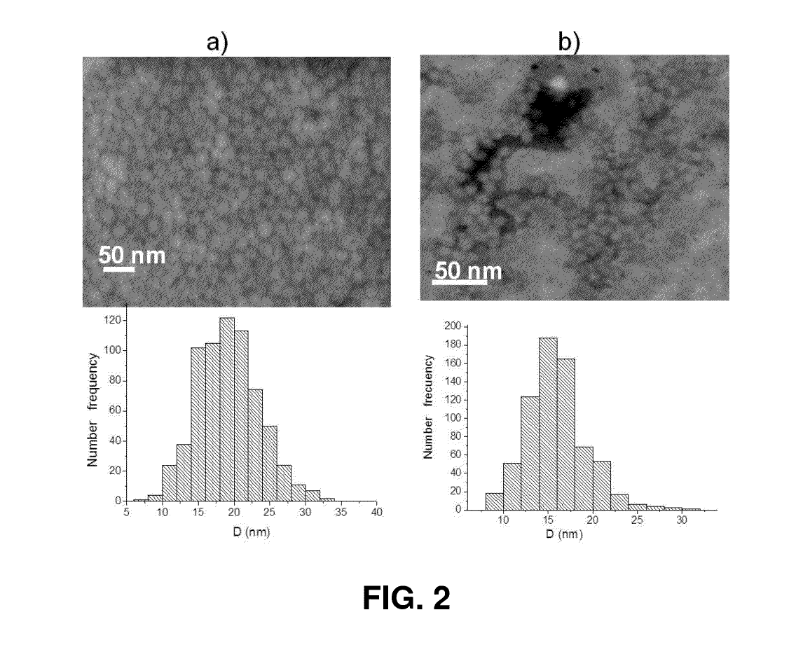

FIG. 2 shows representative micrographs of samples of the filtered latexes from both Example 1 and Example 2 polymerizations, where the spheroidal appearance of nanoparticles is evident. Diameter measurements of around 700 nanoparticles from different micrographs of each of the samples were carried out by using an image analysis program (ImageJ 1.37c). These results were used to construct the corresponding histograms of particle diameters, also included in FIG. 2. In addition, using the data from nanoparticle size measurements, the corresponding D_{w}, Dₙ and PDI values were calculated. Dₙ values of the Ibuprofen-loaded nanoparticles resulting from polymerizations Example 1 and Example 2 (see Table 2) were very small: 19.21 and 15.90 nm, respectively. However, the PDI values (1.14-1.15), also included in Table 2, do not correspond to a particle population with low polydispersity. It is well-known that semicontinuous heterophase polymerization of pure monomer leads to nanoparticles with PDI values usually ≦1.10; quite likely the enhanced PDI values obtained result from the Ibuprofen admixture with the polymerizable monomer. While size monodispersity of drug-loaded polymeric nanoparticles is desirable, we believe the PDI values obtained by us will not diminish the attractiveness of the prepared materials, whose size contrasts with those obtained by other groups who prepared much larger Ibuprofen-loaded poly(methyl methacrylate) microparticles using methods such as solvent evaporation (Sa B, Mondal U K, Prasad N R, Jha T (1996) Development of indomethacin-and-ibuprofen-loaded polymethylmethacrylate microparticles. Pharm Sci 2: 209-213; and Sivakumar M, Rao K P (2002) In vitro release of ibuprofen and gentamicin from PMMA functional microspheres. J Biomater Sci Polym Ed 13: 111-126) and suspension polymerization (Sivakumar M, Rao K P (2002) Synthesis, characterization, and in vitro release of ibuprofen from poly(MMA-HEMA) copolymeric core-shell hydrogel microspheres for biomedical applications. J Appl Polym Sci 83: 3045-3054).

Table 2 also shows the Ibuprofen contents in the nanoparticles. The theoretical contents, calculated based on the recipes and the polymerization conversions, assuming that all the Ibuprofen in the recipe was incorporated into the nanoparticles, were 18.6 and 23.9% of dry basis nanoparticles for Example 1 and Example 2 latexes, respectively. Comparatively, the corresponding values obtained by UV-vis spectrophotometry at 272 nm were 15.7 and 23.1%. Taking into account the experimental error and the precision of the spectrophotometric method, theoretical and spectrophotometric results acceptably match each other. Moreover, these results, along those obtained in the filtration runs, indicate that this method practically operates at 100% of drug entrapment efficiency. This fact is worth highlighting, considering that an efficiency like this is not attained in any of the works reported in the literature. As the lines above indicate, the Ibuprofen content in the nanoparticles obtained by the method of the present invention is not particularly high; however, this potential disadvantage could be overcome through the increase in the Ibuprofen concentration in the solution Ibuprofen-methyl methacrylate dosed during the polymerization.

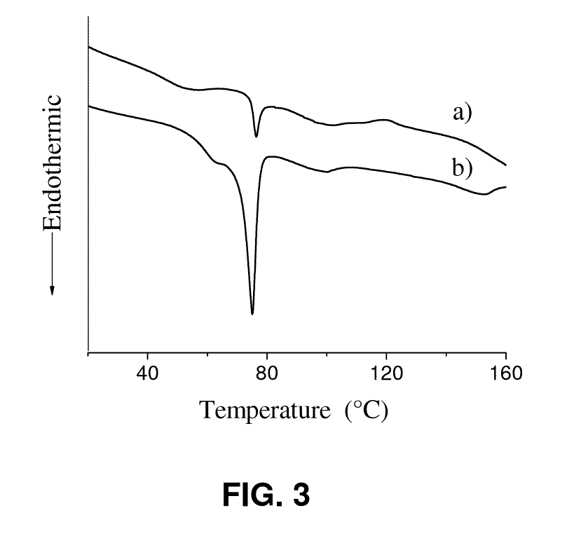

An important factor in determining the drug release rate from the nanoparticles is the physical state of the Ibuprofen inside them. Dispersed molecules would be more easily released from the nanoparticles than crystals of the drug, due to the higher diffusion of the former. From the results obtained by DSC for nanoparticles loaded with different concentrations of Ibuprofen in polystyrene, Dubernet et al. (Dubernet C, Rouland J C, Benoit J P (1991) Ibuprofen-loaded ethylcellulose microspheres: Analysis of the matrix structure by thermal analysis. J Pharm Sci 80: 1029-1033), and Tamilvanan and Biswanath (Tamilvanan S, Biswanath S A (1999) Effect of drug load in the internal structure of ibuprofen-loaded polystyrene microparticles. Acta Pol Pharm 56: 221-226) demonstrated that up to 20% drug loading, the material was dispersed at a molecular level in the polymer, while at Ibuprofen contents ≧30%, solution and crystalline forms coexisted inside the polymer particles. The DSC thermograms for samples of Ibuprofen-loaded nanoparticles from Example 1 and Example 2 polymerizations are shown in FIG. 3. Here, both curves show an endothermic peak close to 75° C., although that corresponding to nanoparticles with the lower Ibuprofen content is less intense. Taking into account that the melting point of Ibuprofen is close to 75° C. (Tamilvanan S, Biswanath S A (1999) Effect of drug load in the internal structure of ibuprofen-loaded polystyrene microparticles. Acta Pol Pharm 56: 221-226), this indicates that even at Ibuprofen concentration (Example 1) lower than 20%, crystals and dispersed molecules of the drug coexist inside the nanoparticles. The difference with that found for Ibuprofen-loaded polystyrene nanoparticles suggest a better compatibility between this polymer and Ibuprofen.

While poly(methyl methacrylate) was selected for probing the model, there are hydrophobic biocompatible polymers, such as poly(vinyl acetate) and some poly(alkyl acrylates) and even biodegradable polymers, such as poly(alkyl cyanoacrylates) that could be used for preparing drug-loaded polymeric nanoparticles by this method. In fact, poly(alkyl cyanoacrylates) can be obtained by polymerization in micelles dispersed in an aqueous continuous phase, such as the ones used in the method of the present invention. Another interesting point is that the surfactant sodium dodecyl sulfate can be replaced by biocompatible surfactants such as methylcellulose, dextrans and poloxamers. Based on the flexibility offered and the method of the present invention, we are confident that the process presented here will find use in the preparation of ultrafine nanoparticles composed of different hydrophobic polymers and water insoluble drugs.

Although the invention was described with reference to specific embodiments, this description is not intended to be built in a limited sense. The different modifications of the embodiments published, as well as alternative embodiments of the invention will be apparent to persons knowledgeable in the state of the art when referring to the description of the invention. For this reason it is considered that the appended claims cover such modifications that fall within the scope of the invention, or their equivalents.

1. A method for producing drug-loaded polymeric nanoparticles comprising the steps of:
preparing a first solution by dissolving a drug in a polymerizable monomer;
preparing a micellar solution by dissolving a surfactant and a water-soluble radical initiator in water;
adding said first solution to said micellar solution for polymerizing said polymerizable monomer, obtaining a dispersion of drug-loaded polymeric nanoparticles, wherein said drug-loaded polymeric nanoparticles have a controlled size with average diameter smaller than 50 nm; and
evaporating residual polymerizable monomer from said dispersion of drug-loaded polymeric nanoparticles.

2. The method of claim 1, wherein said first solution includes:
from 0.1% to 50% by weight of said drug; and
from 50% to 99% by weight of said polymerizable monomer.

3. The method of claim 1, wherein said micellar solution includes:
from 0.1% to 15% by weight of surfactant;
from 0.01% to 10% by weight of water-soluble radical; and
from 85% to 99% by weight of water.

4. The method of claim 1, wherein said drug is selected from a group consisting of Paclitaxel, Docetaxel, Rapamycin, Doxorubicin, Daunorubicin, Idarubicin, Epirubicin, Capecitabine, Mitomycin C, Amsacrine, Busulfan, Tretinoin, Etoposide, Chlorambucil, Chlormethine, Melphalan, Benzylphenylurea (BPU) compounds, curcumin, curcuminoids, flavinoids, natural and synthetic steroids, cyclopamine, Aciclovir, Indinavir, Lamivudine, Stavudine, Nevirapine, Ritonavir, Ganciclovir, Saquinavir, Lopinavir, Nelfinavir, Itraconazole, Ketoconazole, Miconazole, Oxiconazole, Sertaconazole, Amphotericin B, Griseofulvin, quinolones, Ciprofloxacin, Ofloxacin, Moxifloxacin, Methoxyfloxacin, Pefloxacin, Norfloxacin, Sparfloxacin, Temafloxacin, Levofloxacin, Lomefloxacin, Cinoxacin, penicillins, Cloxacillin, Benzylpenicillin, Phenylmethoxypenicillin, aminoglycosides, Erythromycin, macrolides, rifampicin, rifapentin, Ibuprofen, Indomethacin, Ketoprofen, Naproxen, Oxaprozin, Piroxicam, Sulindac, and combinations thereof.

5. The method of claim 1, wherein said drug is Ibuprofen.

6. The method of claim 1, wherein said polymerizable monomer is selected from a group consisting of styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, p-phenylstyrene, methyl acrylate, ethyl acrylate, n-propyl acrylate, iso-propyl acrylate, n-butyl acrylate, iso-butyl acrylate, tert-butyl acrylate, n-amyl acrylate, n-hexyl acrylate, 2-ethylhexyl acrylate, n-octyl acrylate, n-nonyl acrylate, cyclohexyl acrylate, benzyl acrylate, dimethylphosphate ethyl acylate, diethylphosphate ethyl acylate, dibutylphosphate ethyl acylate, and 2-benzoyloxyethyl acrylate, methyl methacrylate, ethyl methacrylate, n-propyl methacrylate, iso-propyl methacrylate, n-butyl methacrylate, iso-butyl methacrylate, tert-butyl methacrylate, n-amyl methacrylate, n-hexyl methacrylate, 2-ethylhexyl methacrylate, n-octyl methacrylate, n-nonyl methacrylate, diethylphosphate ethyl methacylate, dibutylphosphate ethyl methacylate, methylenealiphaticmonocarboxylic acid esters, vinyl acetate, vinyl propionate, vinyl butyrate, vinyl benzoate, vinyl formate, vinyl methyl ether, vinyl ethyl ether, and vinyl isobutyl ether, vinyl methyl ketone, vinyl hexyl ketone, vinyl isopropyl ketone, and combinations thereof.

7. The method of claim 6, wherein said polymerizable monomer is methyl methacrylate.

8. The method of claim 1, wherein said surfactant is selected form a group consisting of sodium dodecylsulfate, dialkylsulfosuccinic acid, sodium bis(2-ethylhexyl)sulfosuccinate, salts of alkyl, aryl sulfonates, salts of tri-chain amphiphilic compounds, sodium trialkyl sulfo-tricarballylates, ethylene oxide, hydroxyl groups, polyoxyethylene alkyl ethers, acetylene diols, alkylthiopolyacrylamides, copolymers of polyoxyethylene and polyoxypropylene, alcohol alkoxylates, sugar-based derivatives, alkyl amines, quaternary ammonium salts, betaines, and combination thereof.

9. The method of claim 8, wherein said surfactant is sodium dodecylsulfate, sodium bis (2-ethylhexyl)sulfosuccinate, and combinations thereof.

10. The method of claim 1, wherein said water-soluble radical initiator is a salt of persulfate anion.

11. The method of claim 10, wherein said salt of persulfate anion is potassium persulfate.

12. The method of claim 1, wherein further said micellar solution includes from 0.01% to 10% by weight of a chain transfer agent selected from a group consisting of selected from a group consisting of dodecanethiol, octadecyl mercaptan, 2-methyl-1-butanethiol and 1,9-nonanedithiol, and combinations thereof.

13. The method of claim 1, wherein said drug-loaded polymeric nanoparticles have a controlled size with average diameter from 5 to 40 nm.

14. A drug-loaded polymeric nanoparticle produced by the method of claim 1.
